(19) United States
(12) Patent Application Publication
(10) Pub. No.: US 2013/0053746 A1
(43) Pub. Date: Feb. 28, 2013

(54) CENTRAL-OPENING KNITTING

(71) Applicants: Sophie Roland, Tignieu (FR); Jean-Roger Guillo, Serezin de la tour (FR)

(72) Inventors: Sophie Roland, Tignieu (FR); Jean-Roger Guillo, Serezin de la tour (FR)

(73) Assignee: ZODIAC AUTOMOTIVE DIVISION, Plaisir Cedex (FR)

(21) Appl. No.: 13/592,680

(22) Filed: Aug. 23, 2012

Related U.S. Application Data
(60) Provisional application No. 61/526,498, filed on Aug. 23, 2011.

(51) Int. Cl. A61F 13/56; B29C 70/22; A61F 13/00; D04B 21/10; B32B 3/10

(52) U.S. Cl. 602/44; 442/1; 428/197; 602/42; 28/169

(57) ABSTRACT

A composite material is provided that includes an open knitted textile support including at least one area free of stitches and/or in which the stitch density is at least five times less than in the rest of the support and a silicone impregnating the support. A method of fabrication of the composite material also is provided, and a medical device including the composite material.

### CROSS-REFERENCE TO RELATED APPLICATION

This application claims priority on U.S. Provisional Patent Appl. No. 61/526,498 filed on Aug. 23, 2011, the entire disclosure of which is incorporated herein by reference.

### BACKGROUND OF THE INVENTION

1. Field of the Invention

The invention concerns a composite material comprising a textile base and intended to be integrated into a medical device, for example of the dressing type. The invention also concerns the method of fabrication of said composite material as well as a medical device comprising it. Finally, the invention concerns the use of a textile-based material in the fabrication of a medical device, for example of the dressing type, or an intermediate product thereof.

2. Description of the Related Art

There are known medical devices that are applied to a skin area to be protected where there is a lesion, for example a dressing. These devices enable the skin area to be protected to be isolated from any exterior contamination by fluids in the environment such as water, by chemical substances such as soap, grease, as well as by bacterial contamination agents or microorganisms. These devices also enable evacuation to the exterior of excess water present inside said device and produced by secretions in the skin area to be protected. Finally, they enable protection of the covered skin area from any form of exterior mechanical aggression.

Such a device generally comprises an absorbent mass enabling secretions of the skin area to be protected, such as exudates from a wound, to be absorbed, an adhesive layer that enables the device to adhere to the skin area around the lesion and to be held in place around that area, and where applicable a covering forming the exterior surface of said device.

Among adhesives, silicones have properties that are particularly interesting: these are “soft” adhesives that have the advantage that removal of the composite material is not painful, as well as encouraging healing.

However, silicone is difficult to use if a thick layer is to be deposited.

There is known from the document WO 87/05206 a dressing intended to be in contact with a wound, comprising an open structure, such as a knitted structure, coated with a hydrophobic soft adhesive such as a silicone.

Thanks to the presence of the soft adhesive, this dressing has the advantage of being well tolerated by the skin and of enabling removal of the medical device without causing pain for the patient.

However, this dressing has the disadvantage of being difficult to use in practice. As it is not able to absorb exudates from the wound, it cannot be employed on its own and must be used in conjunction with an absorbent foam and a tape for attaching it, the combination forming the medical device.

These various components can be assembled only at the time of applying the device, because there is insufficient adhesion between them to enable long-term cohesion; which also necessitates frequent replacement of the medical device.

The object of the present invention is thus to provide a composite material for medical devices intended to come into contact with a wound that may be used with an absorbent mass and that is easy to fabricate and to integrate into a medical device.

### SUMMARY OF THE INVENTION

To this end, the invention concerns a composite material including an open knitted textile support including an array of stitches, said support including at least one area free of stitches or in which the stitch density is at least five times less than in the rest of the support and a silicone impregnating the stitches of said support.

The areas free of stitches or in which the stitch density is at least five times less than in the rest of the support (referred to hereinafter as low stitch density areas) can, when a medical device is used, accommodate an absorbent mass or any other relatively voluminous element.

The material of the invention provides the advantages linked to the use of a soft adhesive (silicone) whilst having good cohesion.

The material of the invention may thus constitute an intermediate product easy to assemble with other intermediate products to obtain a medical device such as a dressing.

The textile support of the invention may include threads enabling it to have a high deformation capacity and a high contention force. For example, the knitted textile includes elastic filaments such as threads including an elastane type polyurethane.

In another example, the knitted textile support includes coated or textured threads.

The composite material of the invention may include one or more identical or different areas free of stitches or of low stitch density that may have a wide variety of shapes. For example, the at least one area free of stitches or of low stitch density has a shape chosen from the following: circular, rectangular, square or oval.

The silicone may impregnate one only or both faces of the support of the material.

The silicone employed is preferably a silicone gel.

In a preferred embodiment of the invention, said silicone is an adhesive silicone. Accordingly, once assembled into a medical device, the material of the invention may be applied directly to the skin.

In one embodiment of the invention, said silicone extends continuously over the surface of the support comprising stitches.

Alternatively, said silicone extends discontinuously over the surface of the support.

The invention also concerns a medical device for protecting a skin area, such as a dressing, including the material described above.

The medical device may typically comprise other intermediate products such as an exterior membrane, an absorbent mass or a perforated tulle type fabric serving as an interface with the skin area to be protected.

The intermediate products in contact with the composite material of the invention may be assembled therewith directly if the silicone impregnating the support is adhesive or by means of an adhesive layer disposed between them.

The medical device of the invention generally includes an exterior membrane.

This membrane permeable to water vapor but impermeable to fluids and to microorganisms then constitutes the external covering of the medical device.

It may be of the breathing or semi-breathing (permeable or semi-permeable) type and produced in diverse materials such as polyurethane, a polyamide, polyethylene terephthalate or a silicone.

The areas free of stitches or of low stitch density of the composite material of the invention enable supplementary elements to be accommodated.

In particular, the medical device of the invention includes an absorbent mass.

This absorbent mass is advantageously disposed in an area free of stitches or in which the stitch density is at least five times less than in the rest of the support of the textile support.

Absorbent masses well known in the field may be used for this purpose, for example a hydrophilic foam based on polyurethane or hydrocolloid fibers.

Where appropriate, notably if the medical device is intended to protect a skin area including a wound, the absorbent mass may be impregnated with an active principle for treating said wound.

The invention also relates to a method of fabricating the composite material described above.

Accordingly, the method of the invention includes the steps consisting in:

knitting on a Jacquard raised hook type device a textile support including areas free of stitches or in which the stitch density is at least five times less than in the rest of the support,

depositing a silicone pre-polymer solution on at least one face of said textile support so as to impregnate said support, and

polymerizing said pre-polymer solution.

The method of the invention is easy to use.

Jacquard knitting techniques are known and well mastered and enable there to be easily obtained a knitted fabric that can feature a great variety of patterns, for example openings (areas free of stitches) or areas of low stitch density of highly varied size and shape.

The step of depositing the pre-polymer solution may be carried out using different processes well known to the person skilled in the art, in particular by coating with a squeegee or cylinder or by pad impregnation.

Depending on the applications envisaged, one or both faces of the knitted fabric may be coated with the silicone pre-polymer solution.

Said method preferably further comprises a step consisting in unblocking the areas free of stitches or in which the stitch density is at least five times less than in the rest of said textile support filled with the pre-polymer solution.

Depending on the viscosity of the pre-polymer solution employed, the areas free of stitches may be blocked, which would make the future integration of an absorbing mass difficult, for example.

The unblocking operation may be carried out in accordance with different techniques, in particular by leaving the pre-polymer solution to drip or blowing a thin layer of compressed air transversely to the direction of movement of the textile layer, with different angles of incidence.

The polymerization step is preferably carried out by introducing the knitted fabric impregnated with the pre-polymer solution into a hot air oven.

A layer of polymerized silicone is then obtained on the surface of the knitted support, which layer may be continuous except in the area free of stitches or having a stitch density at least five times less than in the rest of the textile support.

The quantity of polymerized silicone on the coated face or faces is preferably 50 to 300 g/m².

Once the material has been fabricated, it may be covered with non-stick paper or film to protect it from any aggression and then wound onto a spool.

It is then easily stored and then moved for its integration during the fabrication of a medical device such as a dressing.

The invention relates finally to the use of an intermediate product of the composite material described above in the fabrication of medical devices.

To this end, the invention concerns the use for the fabrication of a medical device for protecting a skin area or for the fabrication of an intermediate product thereof of a material including an open knitted textile support including at least one area free of stitches and/or in which the stitch density is at least five times less than in the rest of the support.

The invention will now be described in more detail with the aid of the appended drawings.

### BRIEF DESCRIPTION OF THE DRAWINGS

FIG. 1 represents a plan view of a material of a first embodiment of the invention.

FIG. 2 represents a plan view of a material of another embodiment of the invention.

FIG. 3 represents a view in section of a medical device of the invention.

FIG. 4 represents a view in section of a variant medical device of the invention.

### DETAILED DESCRIPTION OF THE PREFERRED EMBODIMENTS

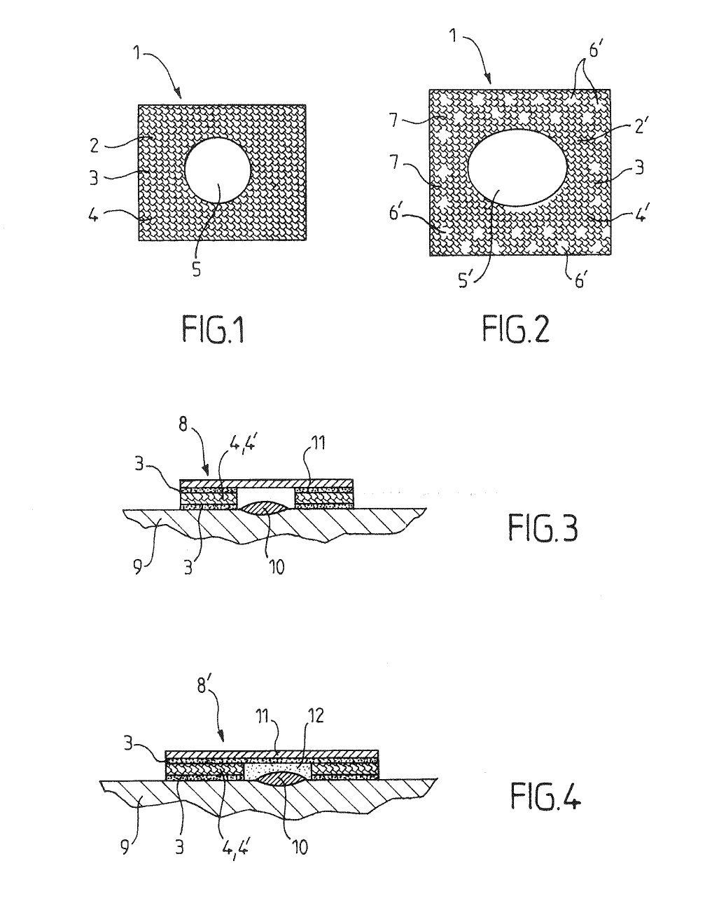

FIG. 1 represents a composite material 1 intended to be incorporated into a medical device comprising a support layer 2 and a silicone gel 3.

The support layer is formed of an array of stitches 4 knitted from an elastic thread enabling the material to offer high capacities for deformation.

Within this support layer is a circular area 5 free of stitches.

The shape of this area 5 free of stitches is adapted to be able where appropriate to receive an element of the medical device into which the material 1 is intended to be integrated, such as an absorbent mass.

The stitches 4 of said support layer 2 are impregnated with an adhesive silicone gel 3 so that the two faces of the support layer 2 are impregnated, there being no silicone gel 3 in the area free of stitches.

The composite material 1 obtained in this way may then be assembled with one or more intermediate products to form a medical device, of which said material will constitute the interface with the skin area to be protected on which the device will be disposed.

FIG. 2 represents an alternative to the material shown in FIG. 1 in which the support layer 2′ formed of an array of stitches 4′ includes not only a circular area 5′ free of stitches but also a plurality of areas 6′ in which the density of the meshes 4′ is at least five less than in the rest of the support layer 2′, so that these areas 6′, because of their low stitch density, form small openings 7 between the stitches 4′.

FIG. 3 represents a medical device 8 of the invention including the composite material 11 shown in FIG. 1.

The medical device 8, here a dressing intended to be supplied to a skin area 9 to be protected including a wound 10, includes the composite material 1 and a semi-permeable membrane 11 assembled together.

The device 8 is disposed so that the composite material 1 comes directly into contact with the skin area to be protected whilst the area 5 free of stitches is situated in front of the wound 10, the adhesive silicone gel 3 enabling a traumatic adhesion to said skin area 9.

The composite material 1 is assembled with a semi-permeable membrane 11, for example based on silicone, constituting the exterior covering of the device 8.

The composite material 1 and the semi-permeable membrane 11 are held together by the adhesive silicone gel 3 impregnating both faces of the material 1.

Thus the dressing 8, while protecting the skin area 9 and the wound 10, enables evacuation of exudates produced by the wound via the semi-permeable membrane 11.

FIG. 4 shows an alternative to the medical device shown in FIG. 3, in which the device 8′ comprises the composite material 1 and a semi-permeable membrane but also an absorbent mass 12 disposed within the area 5 free of stitches.

Thus the absorbent mass comes directly into contact with the wound 10 and absorbs exudates generated by the wound.

This variant is particularly suitable when the skin area to be protected includes an exudating wound and makes it possible to prevent the exudates accumulating on the surface of the wound, impeding healing.

What is claimed is:

1. A composite material (1, 8, 8′) including an open knitted textile support (2, 2′) including an array of stitches (4, 4′), said support including at least one area (5, 5′ and/or 6′) free of stitches in which the stitch density is at least five times less than in the rest of the support and a silicone (3) impregnating said support.

2. The material of claim 1, wherein the knitted textile support (2, 2′) includes elastic filaments.

3. The material of claim 1, wherein the knitted textile support (2, 2′) includes coated or textured threads.

4. The material of claim 1, wherein the at least one area free of stitches or in which the stitch density is at least five times less than in the rest of the support has a shape chosen from the following: circular, rectangular, square or oval.

5. The material of claim 1, wherein said silicone is an adhesive silicone.

6. The material of claim 1, characterized in that said silicone extends continuously over the surface of the support.

7. The material of claim 1, characterized in that said silicone extends discontinuously over the surface of the support.

8. A medical device for protecting a skin area, including the material of claim 1.

9. The medical device of claim 8, further comprising an absorbent mass (12) disposed in the area free of stitches or in which the stitch density is at least five times less than in the rest of the support of the textile support.

10. The medical device of claim 8, further comprising an exterior membrane (11).

11. A method of fabricating the composite material of claim 1, including the steps of:
knitting on a Jacquard raised hook device a textile support including areas free of stitches or in which the stitch density is at least five times less than in the rest of the the support,
depositing a silicone pre-polymer solution on at least one face of said textile support so as to impregnate said support, and
polymerizing said pre-polymer solution.

12. The method of claim 11, characterized in that the step of depositing the pre-polymer solution is carried out by coating with a squeegee or roller or by pad impregnation.

13. The method of claim 11, further comprising unblocking openings of said textile support filled with the pre-polymer solution.
